(19) United States
(12) Patent Application Publication
(10) Pub. No.: US 2006/0234214 A1
(43) Pub. Date: Oct. 19, 2006

(54) Methods of detecting hepatitis C virus

(76) Inventor: Kye-Hyung Paik, Rancho Santa Fe, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/405,032

(22) Filed: Apr. 13, 2006

Related U.S. Application Data
(60) Provisional application No. 60/671,695, filed on Apr. 15, 2005.

(51) Int. Cl. C12Q 1/70; C12P 21/04; C07K 14/18; C07K 16/10

(52) U.S. Cl. 435/5; 530/350; 530/388.4; 435/70.21

(57) ABSTRACT

The present invention provides a sensitive and specific test for hepatitis C virus. The test detects the presence of particular core antigens of hepatitis C, which appear earlier after initial infection than antigens or viral genetic materials used in previously-developed assays. The invention test allows detection of hepatitis C infection at an earlier stage of infection with a lower number of false positives than previously-developed tests. The invention also provides a novel core protein antigen and novel antibodies to the core protein antigen.

This application claims the benefit of priority of U.S. provisional application Ser. No. 60/671,695, filed Apr. 15, 2005, the entire contents of which is incorporated herein by reference.

### BACKGROUND OF THE INVENTION

1. Field of the Invention

The present invention relates generally to biological assay methods and, in particular, to methods of detecting hepatitis C virus.

2. Background

Hepatitis C virus (HCV) is the causal agent for a largely chronic liver infection originally identified as non-A, non-B hepatitis. The virus was first identified by expression cloning of the genome from a serum sample of a chimpanzee using an acute non-A non-B patient serum for detection. HCV has infected about four million people in the United States and 170 million worldwide, about four times as many as HIV. The number of deaths from HCV is now 8,000 to 10,000 annually in the United States and is considered likely to triple by 2010. In addition, HCV infection is one of the primary causes of liver transplantation in the United States and other countries. Post-transfusion hepatitis (PTH) occurs in approximately 10% of transfused patients and HCV accounts for up to 90% of these cases. The disease frequently progresses to chronic liver damage (25-55%) (Rosenberg, J. Mol. Biol. 2001; 313 451-464).

HCV is a RNA virus of the Flaviviridae, genus Hepacivirus, and is most closely related to the pestiviruses, BVDV and GBV-B. The HCV genome is composed of a single positive strand of RNA, approximately 9.6 kb in length. The HCV genome possesses a continuous, translational open reading frame (ORF) that encodes a protein of about 3,000 amino acids. The structural protein(s) appear to be encoded in approximately the first quarter of the N-terminus region of the ORF, the remainder coding for non-structural proteins. In addition to the translated region, untranslated regions are present in the HCV genome. The 5′ untranslated region (UTR) contains the most highly conserved sequence among all HCV isolates and seems to consist of important regulatory elements. The 5′ UTR is composed of the transcriptional initiation site for positive-stranded viral RNA synthesis as well as an unusual internal ribosome entry site (IRES) that plays a role for viral polyprotein translational initiation. The 3′ untranslated regions have a variable sequence of approximately 40 bases, a variable length poly-UC rich tract and a highly conserved 98 base region. It is believed that both viral and host proteinases process the polyprotein of 3,000 amino acids from the remainder of the viral RNA. The HCV polyprotein comprises, from the amino terminus to the carboxy terminus, the nucleocapsid protein (C), the envelope protein (E1 and E2), p7 and the non-structural proteins (NS) 2,3,4A, 4B,5A and 5B.

HCV is responsible for a transmissible disease distinguishable from other forms of viral-associated liver diseases, including those caused by the hepatitis viruses: hepatitis A virus (HAV), hepatitis B virus (HBV), and delta hepatitis virus (HDV), as well as the hepatitis induced by cytomegalovirus (CMV) or Epstein-Bar virus (EBV). Six major genotypes of HCV are present worldwide with sequence differences up to 30%.

The present treatments for HCV infection are alpha-interferon in combination with ribavirin or a polyethylene glycol modified form of alpha-interferon. Response rates with these treatments are modest and show significant variation depending upon the specific HCV genotype.

It is presumed that the primary route of infection is through contact with contaminated bodily fluids, especially blood, from infected individuals. Thus, the increasing prevalence of HCV infection poses a serious risk to the supply of blood from anonymous don\dot{o}rs. There is thus significant demand for sensitive, specific methods for identifying carriers of HCV and screening of HCV contaminated blood or blood products. Early and accurate detection of HCV infection is necessary in order to ensure effective patient care and to ensure that adequate measures are undertaken to prevent HCV transmission.

The most sensitive conventional HCV diagnostic tests available to date are approximately 98-99% accurate. Even a 1-2% error rate results in an unacceptably high risk to the integrity of the blood supply. Likewise a lower error rate would aid in patient care. There is thus a need for more sensitive and accurate methods of detecting hepatitis C virus in biological samples. The present invention meets this need and provides related advantages as well.

### SUMMARY OF THE INVENTION

The present invention provides a method of detecting hepatitis C virus by detecting a mitochondrially expressed HCV peptide.

The invention further provides an isolated peptide consisting of the amino acid sequence represented by SEQ ID NO:3, wherein Xaa is Arg or another amino acid residue. Specifically, the invention provides a mitochondrially expressed HCV peptide of SEQ ID NO:3, especially of SEQ ID NO:4.

Additionally, the invention provides a method of making an antibody to a mitochondrially expressed peptide of SEQ ID NO:3. The method includes introducing a mitochondrially expressed HCV short core peptide of SEQ ID NO:3 into an animal and isolating splenocytes from the animal. The splenocytes are then fused to immortalized cells to form hybridomas. Antibody to the mitochondrially expressed HCV peptide of SEQ ID NO:3 is then isolated from hybridomas that test positive for the antibody.

Furthermore, the invention provides a method of detecting HCV. The method includes detection of a mitochondrially expressed peptide consisting of the amino acid sequence of SEQ ID NO:3.

The invention also provides a method of detecting HCV in a sample. The method is a commonly known as a sandwich assay and includes providing a support-bound antibody that binds to an epitope of a mitochondrially expressed HCV peptide of SEQ ID NO:3. Binding of the peptide to the antibody results in an immobilized antibody-antigen complex. This antibody-antigen complex can be detected by contacting it with a labeled antibody to a second epitope on the mitochondrially expressed HCV peptide of SEQ ID NO:3. Labeled antibody that binds to the peptide can be detected by a conventional method for detecting the label. Detection of bound labeled antibody indicates the presence of mitochondrially expressed HCV peptide of SEQ ID NO:3 in the sample. Where the sample is derived from a subject, such as a human subject, detection of the mitochondrially expressed HCV peptide of SEQ ID NO:3 in the sample indicates that the subject is infected with HCV. Thus, the invention also provides a method of diagnosing a subject with HCV by detecting the presence of mitochondrially expressed HCV peptide of SEQ ID NO:3 in a sample from the subject.

The invention further provides a kit useful for detecting HCV in a sample or HCV infection in a subject. Such a kit includes two antibodies, each of which binds to an epitope of a mitochondrially derived HCV peptide of SEQ ID NO:3. In some embodiments, the first antibody is bound to a support, such as a bead or a well of a test plate, while the second antibody is detectably labeled. In some embodiments, the kit also includes one or more additional solutions, such as a rinsing solution, a solution containing a reagent for detecting the label, or both.

A method of detecting HCV, comprising detecting a mitochondrially expressed peptide consisting of the amino acid sequence of SEQ ID NO:4.

### BRIEF DESCRIPTION OF THE DRAWINGS

FIG. 1 shows the peptide sequence of the hepatitis C virus (HCV) core protein (SEQ ID NO:1) and the sequence of the HCV RNA encoding the core protein (SEQ ID NO:2).

FIG. 2 shows the peptide sequence of hepatitis C virus short core protein (SEQ ID NO:3) and indicates a first claimed peptide, wherein Xaa is Arg or another amino acid residue.

FIG. 3. shows the peptide sequence of a particular hepatitis C virus short core protein (SEQ ID NO:4). This sequence corresponds to positions 1-39 of SEQ ID NO:1.

FIG. 4 is a schematic representation of mitochondrial expression of HCV peptides and the early appearance of mitochondrially expressed peptides in blood.

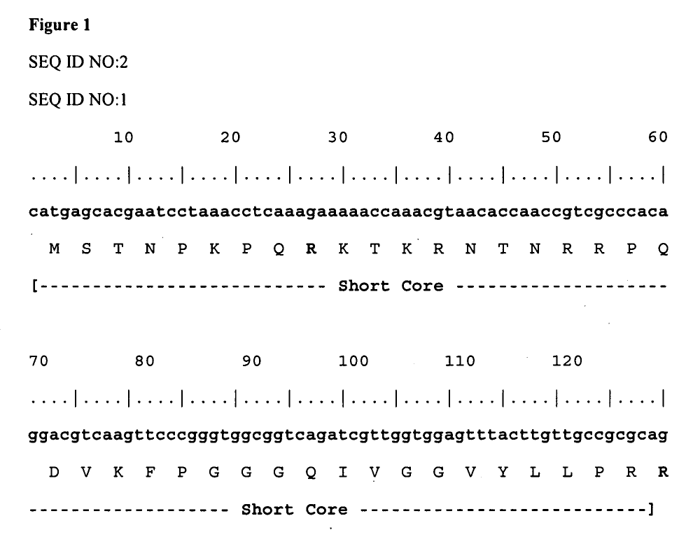

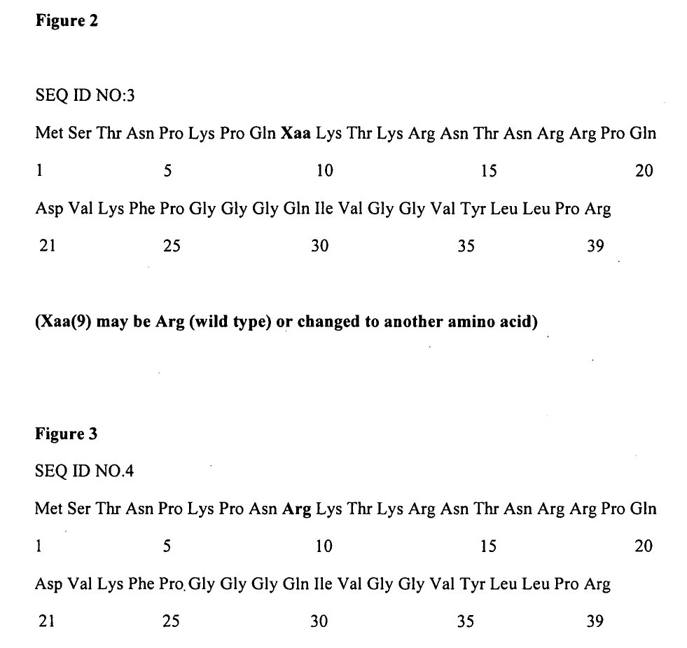

### DETAILED DESCRIPTION OF THE INVENTION

The invention provides methods for detecting hepatitis C virus (HCV) as well as peptides useful for making antibodies to mitochondrially expressed HCV peptides, the antibodies to mitochondrially expressed HCV peptides and kits containing antibodies and optionally other reagents for carrying out the HCV detection methods.

The method includes detection of at least one mitochondrially expressed HCV peptide in a sample. The sample may be blood or another biological fluid or tissue derived from a subject. The method of the invention is surprisingly sensitive and accurate as compared to previously known methods of detecting HCV, because mitochondrially expressed peptides are expressed early, are individually soluble, and are in many cases detectable in biological fluids such as blood before intact virions and even before HCV RNA.

The method of the invention relies upon detection of a mitochondrially expressed HCV peptide. The mitochondrial expression system uses different transfer RNA (tRNA) and different codons from the universal (also known as genomic: that is nuclear and cytoplasmic) expression system, the peptides expressed from HCV RNA in the mitochondria differ in primary structure from those expressed in the nucleus and cytoplasm. In particular cases, the mitochondrial expression system recognizes certain codons, which code for Arg in the universal coding system, as stop codons. Accordingly, mitochondrially expressed HCV peptides are generally short, soluble and not associated with intact virions. They thus make excellent antigens for monoclonal antibodies in that they do not generally need to be solubilized with detergent and thus provide excellent epitopes for antibody binding.

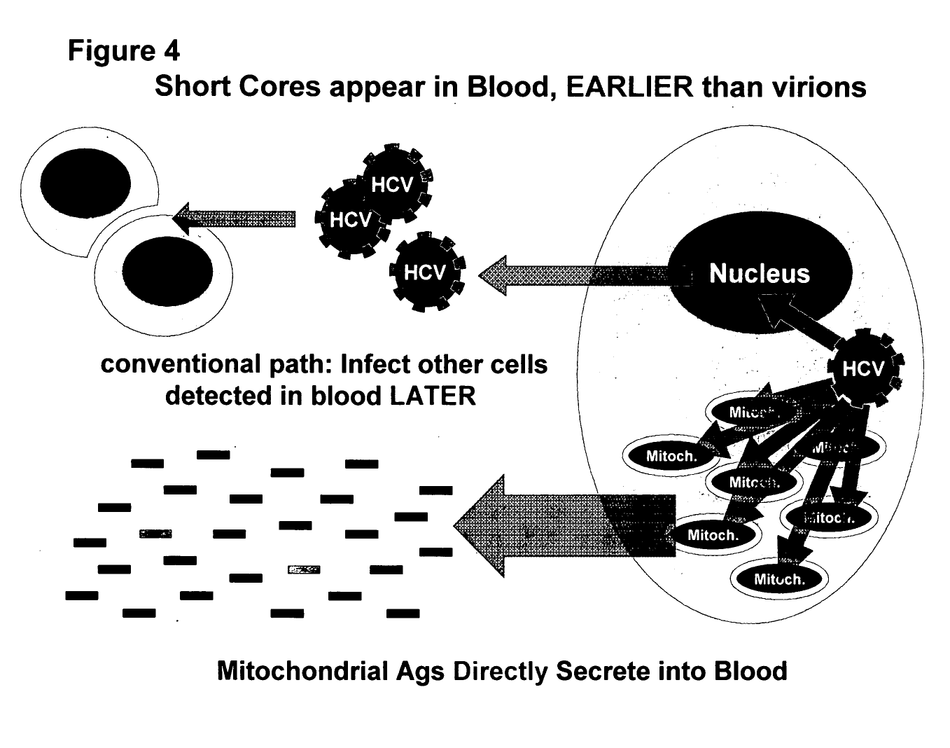

As shown schematically in FIG. 4, it has been surprisingly found that mitochondrially expressed HCV peptides appear in biological fluids, such as blood, earlier than complete virions and even before intracellularly replicated HCV RNA. It has thus been discovered that a HCV assay of the invention, which includes detecting mitochondrially expressed HCV peptides, is capable of detecting HCV contamination earlier in the infection cycle than is possible with previously known methods, such as those immunological methods of detecting intact virion and methods of detecting HCV RNA (such as polymerase chain reaction). Additionally, the sensitivity and accuracy of the methods of the invention are superior to the previously known methods. Moreover, because mitochondrially expressed peptides appear in the blood earlier than HCV RNA, the method of the present invention provides surprisingly superior HCV blood screening and early detection for aggressive antiviral treatment for cure.

In some embodiments of the invention, the HCV detection method comprises detecting only a mitochondrially expressed HCV peptide of SEQ ID NO:3, wherein Xaa is Arg or another amino acid residue. In particular embodiments, the mitochondrially expressed HCV peptide of SEQ ID NO:3 is represented by SEQ ID NO:4.

In some embodiments, the methods of the invention comprise detection of a single mitochondrially expressed HCV peptide of SEQ ID NO:3, 4.

In particular, one such antibody is directed toward a first epitope on a mitochondrially expressed HCV peptide of SEQ ID NO:3, 4, and is bound to a support, such as a solid support comprising a bead or a surface of a sample plate. In such cases, the second antibody is conjugated to a label, such as a fluorescent label, a radiolabel or an enzyme that is capable of catalyzing a detectable chemical reaction in the presence of a suitable reagent.

Since it has been surprisingly found that mitochondrially expressed HCV peptides are present in the blood stream before intact virions, and often before HCV RNA can be detected in the blood stream, the methods of the invention are particularly useful for detecting HCV in samples derived from subjects who are infected with HCV but are asymptomatic of HCV infection. In particular embodiments of the invention, the method includes detecting only one mitochondrially expressed HCV peptide of SEQ NO:3, wherein Xaa is Arg or another amino acid. In particular embodiments, the mitochondrially expressed HCV peptide of SEQ ID NO:3 is the mitochondrially expressed HCV peptide of SEQ ID NO:4. Because these peptides are short, soluble and transcribed early in the HCV infection cycle and soluble, they appear in biological fluids early in the infection cycle. Thus, the methods of the invention are highly sensitive assays useful for the detection of HCV contamination in blood and other bodily fluids. Accordingly, the methods of the invention may be used, in particular, in the screening of blood for transfusion, especially where a subject from which the sample is taken is asymptomatic for HCV infection.

In other embodiments, the methods of the invention may be used, in particular, in early detection of HCV infection for aggressive treatment for cure, especially where a subject from which the sample is taken is asymptomatic for HCV infection.

The invention provides novel peptides of HCV core protein. The novel peptides correspond to mitochondrially expressed HCV peptides. In some embodiments of the invention, a novel peptide of the invention is the HCV short core peptide (SEQ ID NO:3), which represents amino acids 1-39 of SEQ ID NO:2, wherein Arg(9) of SEQ ID NO:1 is replaced by Xaa(9), which is Arg or another amino acid. In certain embodiments, the short core peptide of SEQ ID NO:3 is the peptide of SEQ ID NO:4. In other embodiments, the amino acid represented by Xaa(9) is an amino acid other than Arg.

It has been surprisingly found that an assay employing a monoclonal antibody to the HCV short core peptide (SEQ ID NO:3, 4) surpasses the accuracy and sensitivity of conventional HCV detection assays, because these peptides are mitochondrially expressed.

As used herein the term “HCV short core” refers to SEQ ID NO:3, which corresponds to amino acids 1-39 of SEQ ID NO:1, wherein Xaa is Arg or another amino acid. In particular embodiments, the term “HCV short core” refers to a compound having a peptide portion that consists of SEQ ID NO:3, in particular SEQ ID NO:3, wherein Xaa is Arg. The HCV short core peptide may be covalently linked to other, non-peptidyl portions, including, for example, sugars and artificially-introduced labels, without interfering with the purpose of the present invention.

The terms “epitope” and “antigen” are used synonymously herein to indicate a chemical structure to which an antibody will bind. In general, the epitopes of the invention are peptides, that is chemical compounds including a plurality of amino acid subunits joined to one another by amide bonds. It is also possible for a compound possessing an epitope of the invention to possess at least some non-peptidyl character. For example, an epitope of the invention may, in addition to the specific amino acid sequences identified herein include a covalently immunological adjuvant, a bound sugar, label or solid substrate.

Replication of HCV has been associated with cellular mitochondria. Mitochondria are multilayer membranous cellular organelles that grow and divide in a coordinated process that requires contributions from the genetic system in the nucleus of the cell and the separate genetic system contained in the mitochondria (Alberts et al., Molecular Biology of The Cell, 2nd Ed., pp. 387-401, Garland Publishing, Inc., New York, N.Y.). Most mitochondrial proteins are encoded by nuclear DNA that is transcribed and translated in the cytosol and then imported into the mitochondria. However, a certain percentage of the mitochondrial proteins are transcribed from mitochondrial DNA (mtDNA) and translated within the organelle itself using a non-universal genetic code. The mitochondrial system includes two ribosomal RNA and 22 tRNAs. Comparison of the mitochondrial gene sequences with the amino acid sequences of the encoded proteins reveals that the genetic code within mitochondria is altered compared to the universal code used in the nucleus of eukaryotic cells and in most prokaryotes. For example, the UGA codon is a stop codon for protein synthesis in the universal code whereas UGA codes for tryptophan in mitochondria, and the codons AGA and AGG code for arginine in the universal system but are stop codons in mammalian mitochondria.

The peptides of the invention, such as the HCV short core peptide (SEQ ID NO:3, wherein Xaa(9) is Arg or another amino acid; specifically SEQ ID NO:4), is produced in the mitochondria of an infected host cell. Mitochondrial expression results in different peptides from expression in the nucleus or cytosol, as the mitochondria use a different coding system from the universal system employed in the nucleus and cytosol. Because of this different coding system, which can read Arg codons as stop codons, mitochondrial expression results in the production of soluble polypeptides. It has been found that soluble polypeptides provide excellent epitopes for production of, and detection by, antibodies. In particular, soluble HCV peptides expressed in the mitochondria appear in blood earlier than intact HCV virions. Thus, the mitochondrially-expressed HCV short core peptide (SEQ ID NO:3; SEQ ID NO:4) is superior peptides for the production of antibodies, detection methods and kits for the detection of HCV.

As the peptides of the invention are present on the N-terminal portion of the 3,000 amino acid HCV polyprotein, they are produced early in the HCV replication cycle. It has been discovered that the peptides of the invention are present in the blood plasma of an infected host very soon after the host has been infected with HCV. Accordingly, detection of these peptides offers an advantage over conventional HCV test methods, as they can be detected earlier and with greater sensitivity than can peptides that are detected by conventional tests.

The production of peptides, such as the HCV short core peptide (SEQ ID NO:3, SEQ ID NO:4), is known in the art Thus, the person skilled in the art will recognize that the peptides of the invention can be produced by either chemical synthetic or recombinant methodologies. Peptides of the invention may be produced in a mitochondrial expression system, such as that set forth in U.S. Pat. No. 6,100,068, which is expressly incorporated herein by reference. In general, such a method involves introducing into the mitochondria of a cell the genetic material to be expressed in the mitochondria. Tissue from an organ rich in mitochondria is first provided and the tissue is infected in vitro with a virus, such as a recombinant hepatitis B virus (HBV) containing the genetic code for the desired peptide (epitope). The tissue is then cultured to produce viral peptides through the mitochondrial translation system in the tissue. Finally the expressed peptide is isolated and used to produce antibodies as described herein.

Peptides of the invention may also be produced by chemical synthetic methods. It is known, for instance, to produce peptides by solid phase synthesis on a suitable support medium, for example by the method pioneered by Merrifield (J. Am. Chem. Soc. 91:501-502 (1969)). Such methods are especially suitable for producing relatively short peptides, such as the HCV short core peptide (SEQ ID NO:3).

The HCV short core peptide SEQ ID NO:3, or SEQ ID NO:4 may also be produced by recombinant methods in a standard expression system, such as a bacterial expression system. In such methods, a nucleic acid coding for the desired epitope is cloned into an expression vector, such as a plasmid or a virus. The plasmid or virus is then introduced into a host cell, which expresses the desired peptide. The desired peptide is then separated from the host cell and purified by standard methods. In the present case, the person skilled in the art will recognize that the coding regions of SEQ ID NO:2 that correspond to the peptide to be produced (SEQ ID NO:3, 4) will produce the desired peptide in a mitochondrial expression system as described above. In order to ensure that the proper peptide is produced in the universal system, it will be necessary to make appropriate changes to the coding sequence where the universal and mitochondrial coding systems diverge. Thus, the person skilled in the art will recognize that nucleotides 1-117 of SEQ ID NO:2 code for amino acids 1-39 of SEQ ID NO:1 (SEQ ID NO:3 (upon changing one stop codon at amino acid 9 to another codon, such as one encoding Arg) or SEQ ID NO:4) in the mitochondrial system. Where appropriate, the person skilled in the art will make the appropriate changes to the nucleic acid code such that the HCV peptide corresponding to a SEQ ID NO:3, 4 will be encoded in the universal coding system, and will thus be expressed in the host cells.

The invention also provides antibodies, especially monoclonal antibodies (MAb) that specifically bind to the HCV short core peptide (SEQ ID NO:3 (wherein Xaa is Arg or another amino acid) or SEQ ID NO:4). It has been surprisingly found that antibodies, and especially monoclonal antibodies, that specifically bind the HCV short core peptide are especially suitable for use in assays for the detection of HCV. Assays using monoclonal antibodies to the HCV short core peptide are capable of exceeding the best previously known assays in accuracy and sensitivity.

As the HCV short core peptides are the earliest produced of the HCV peptides, assays using the monoclonal antibodies to the HCV short core peptide are expected to provide the earliest possible detection of HCV.

“Specific” binding indicates that the antibodies of the present invention do not substantially cross-react with other components of HCV or other proteins. Binding of an antibody of the invention to HCV short core peptide can be demonstrated by an art-recognized method such as binding assays, Western Blot assays, ELISA (enzyme-linked immunosorbent assay) assays, RIA-type assays or competition assays.

The term “monoclonal antibody” or “MAb” used herein refers to an antibody composition having a homogeneous antibody population. The term is not limiting regarding the species or source of the antibody itself, nor is it intended to be limited by the manner in which it is made. In addition, the term “antibody” also refers to humanized immunoglobulin sequences and single chain antibodies as described in U.S. Pat. No. 4,946,778 and to fragments of antibodies such as F_{ab}, F_{'ab2}, Fᵥ and other fragments which retain the antigen binding function and specificity of the parent antibody.

Antibodies to be prepared according to a method of the invention include monoclonal antibodies that specifically bind to the peptides of the invention. A monoclonal anti-HCV short core peptide antibody of the invention as described further below, can be used in a variety of applications. Such applications include, for example, detection of HCV infection in a sample or in a subject suspected of being infected with HCV from which a sample is derived. As used herein, the term “antibody” includes monoclonal antibodies as well as polypeptide fragments of monoclonal antibodies that selectively bind to an peptide of the present invention. Such selective binding refers to the discriminatory binding of the antibody to the indicated target peptide such that the antibody does not substantially cross react with unrelated antigens. Specific reactivity can include binding properties such as binding specificity, binding affinity and binding avidity. For example, an antibody can bind a target peptide or polypeptide with a binding affinity (Kd) of about 10⁻⁴ M or more, 10⁻⁶ M or more, 10⁻⁷ M or more, 10⁻⁸ M or more, 10⁻⁹ M or more, or 10⁻¹⁰ M or more.

Methods of producing a monoclonal antibody are well known (see, for example, Harlow and Lane, supra, 1988). For example, mice, such as Balb/c, are immunized with the appropriate immunogen (that is HCV short core peptide (SEQ ID NO:3, wherein Xaa is Arg or another amino acid or SEQ ID NO:4) or HCV prime core peptide (SEQ ID NO:4)). The immunogen is dissolved in complete Freund's adjuvant; then a suitable dose of immunogen (such as about 1-100 μg) is injected subcutaneously or intraperitoneally into the animal. Alternatively the immunogen is emulsified in an adjuvant and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect raised antibodies.

After a suitable antibody titer is detected, the animals with the antibody reactivity can be injected with a final intravenous injection of the immunogen. Three to four days later, the mice are sacrificed and the spleen cells harvested. The spleen cells are then fused (using 35% polyethylene glycol) to a selected murine myeloma cell line. The fusion generates hybridoma cells, which can then be plated in 96 well tissue culture plates containing hypoxanthine, aminopterin and thymidine (HAT) medium to inhibit proliferation of non-fused cells, myeloma hybrids and spleen cell hybrids.

The hybridoma cells are then screened in an ELISA for reactivity against the peptide of the invention (SEQ ID NO:3, wherein Xaa is Arg or another amino acid, or SEQ ID NO:4). The hybridoma cells with positive reactivity can then be injected intraperitoneally into syngenic Balb/c mice to produce ascites containing the monoclonal antibody. Purification of the monoclonal antibodies in the ascites can be performed using ammonium sulfate precipitation, followed by gel exclusion chromatography. Alternatively, affinity chromatography based upon binding of antibody to protein A or protein G can be used.

A variety of well-known methods can be used for collecting from an animal a sample containing an antibody or antibody-producing cell. Such methods are described, for example, in Harlow et al., supra, 1998. Similarly, a variety of well known methods can be used for processing a sample to isolate a anti-HCV short core peptide (SEQ ID NO:3, wherein Xaa is Arg or another amino acid, or SEQ ID NO:4) antibody. A procedure for collecting an processing a sample can be selected based on the type of antibody to be isolated. As an example, when isolating monoclonal antibodies, an appropriate sample can be an antibody-producing cell such as a spleen cell. Exemplary well known procedures for isolating both monoclonal and polyclonal antibodies are known in the art as described above.

The present invention also provides, in particular embodiments, antibody pairs. In particular embodiments, one antibody is bound to a support, such as an insoluble support, while a second antibody is bound to a label, such as a fluorescent label, a radiolabel or an enzyme capable of catalyzing a detectable transformation of a detection reagent. In such embodiments, both antibodies are directed toward the same mitochondrially expressed HCV peptide of SEQ ID NO:3, or SEQ ID NO:4, wherein in SEQ ID NO:3, Xaa is Arg or another amino acid. Such antibody pairs are especially well-suited for methods of the present invention, as they recognize mitochondrially expressed HCV compounds that are expressed early in the HCV infection cycle, are soluble and are thus found in biological tissues of subjects infected with HCV, and in particular HCV-infected subjects who are asymptomatic of HCV infection.

The present invention also provides assay methods capable of determining the presence of HCV short core peptide (SEQ ID NO:3 or SEQ ID NO:4) in a sample, such as a biological sample. The method includes obtaining an anti-HCV short core peptide antibody as described above and contacting a sample to be tested with the anti-HCV short core peptide antibody. The contacting step is carried out under conditions suitable for the antibody to bind the HCV short core peptide (SEQ ID NO:3 or SEQ ID NO:4) to form an immunological complex. Unbound antibody and peptide are then separated from antibody-peptide complex and the complex is detected. Detection of the complex, such as by fluorescence, RIA, Western Blot or ELISA, includes qualitative or quantitative detection.

In a particular embodiment, a method of the invention is a sandwich assay. The method employs an antibody pair. In particular embodiments, one antibody is bound to a support, such as an insoluble support, while a second antibody is bound to a label, such as a fluorescent label, a radiolabel or an enzyme capable of catalyzing a detectable transformation of a detection reagent. In such embodiments, both antibodies are directed toward the same mitochondrially expressed HCV peptide of SEQ ID NO:3, or SEQ ID NO:4, wherein in SEQ ID NO:3, Xaa is Arg or another amino acid. In the sandwich method according to the invention, a first support-bound antibody is provided. A sample is contacted with the support-bound antibody, whereby mitochondrially expressed HCV peptide in the sample binds to the support-bound antibody to form an antibody-antigen complex, which is bound to the support. The support-bound antibody-antigen complex is then contacted with a second antibody that is labeled. The labeled antibody, which binds the same mitochondrially expressed HCV peptide as the first antibody (albeit at a separate epitope) forms with the support-bound antibody-antigen complex a labeled antibody-antigen-antibody sandwich. After removal of unbound labeled antibody, the bound labeled antibody is detected by a suitable method. For example, a bound radiolabel may be quantitated via scintillation counting or Geiger counting. A fluorescent label may be quantitated with a spectrophotometer. An enzymatic label (such as used in ELISA sandwich assays) may be quantitated using a reagent that is transformed to a colored species having a specific absorption spectrum, which can be measured with a spectrophotometer after incubating the labeled antibody-antigen-antibody complex in the presence of the reagent for a period of time. Such methods using antibody pairs directed toward the same mitochondrially expressed HCV peptide are especially useful, as they detect mitochondrially expressed HCV compounds that are expressed early in the HCV infection cycle, are soluble and are thus found in biological tissues of subjects infected with HCV, and in particular HCV-infected subjects who are asymptomatic of HCV infection.

Methods of the invention can be used to detect HCV in a sample, or to detect HCV infection in a subject by detecting the presence of mitochondrially expressed HCV peptide in a sample (such as blood, blood plasma or a tissue biopsy) derived from the subject.

The invention likewise provides assay methods capable of determining the presence of HCV short core peptide (SEQ ID NO:4) in a sample, such as a biological sample. The method includes obtaining an anti-HCV short core peptide antibody as described above and contacting a sample to be tested with the anti-HCV short core peptide antibody. The contacting step is carried out under conditions suitable for the antibody to bind the HCV short core peptide (SEQ ID NO:3, 4) to form an immunological complex. Unbound antibody and peptide are then separated from antibody-epitope complex and the complex is detected. Detection of the complex, such as by fluorescence, RIA or ELISA, includes qualitative or quantitative detection. Such methods are especially useful in histological methods for detecting HCV in a tissue sample, such as a tissue biopsy.

The invention further provides methods of detecting HCV in a sample, comprising detection of mitochondrially expressed HCV peptide with amino acids 1-39.

The terms “sample” and “test sample” refer to any biological material obtained from a subject, such as serum, plasma, saliva, mucus, spinal cord fluid, or biopsies. The term “biopsy” particularly refers to a sample comprising cells, in particular human cells. Moreover, the latter term refers also to a sample derived from liquid tissue, such as peripheral blood cells, or from solid tissue, such as liver tissue. In particular embodiments of the invention, the sample is blood plasma, in particular blood plasma derived from one or more human subjects.

The term “subject” refers to any animal, especially a mammal, including a human, from which a sample is obtained. A subject may, but need not necessarily, be a patient. A negative control sample may be obtained from a subject known to be free of HCV infection, while a positive control sample may be obtained from a subject known to be infected with HCV.

“Detection” means art-recognized immunological methods for detecting antibody-antigen binding, including RadioImmunological Assay (RIA), Enzyme-Linked ImmunoSorbent Assay (ELISA), fluorescence assays, Western Blot assays, etc.

ELISA is well-known in the art of immunology. In general, an enzyme capable of converting a reagent from a colorless to a colored form is coupled to an antibody. Enzymes that have been used successfully in ELISA include alkaline phosphatase, horseradish peroxidase, p-nitrophenyl phosphatase, etc. Enzyme binding is detected by covalently linking a suitable enzyme to an antibody to form an enzyme-antibody conjugate. The conjugate is then contacted with a sample containing an antigen (such as, for example, a peptide of SEQ ID NO:3 or 4) to form an antibody-antigen complex. After separating the complex from unbound antibody and antigen, the complex is detected by adding a reagent which, in the presence of the enzyme, is converted from an uncolored to a colored form. Quantitative or qualitative measurement of the color change provides a measure of the amount of antigen in the sample.

There are various types of ELISA methods available to the person skilled in the art. Some that may be mentioned include indirect ELISA, sandwich ELISA, and competitive ELISA. In sandwich ELISA, for example, antibody to an antigen is bound to a substrate. Such binding is commonly covalent, but it may also be adsorptive. A test sample containing the antigen is contacted with bound antibody. After incubation for a period sufficient to achieve binding of the antigen to the antibody, a solution containing antibody coupled to a suitable enzyme (antibody-enzyme) is brought into contact with the bound antibody-antigen complex. Binding of the antibody-enzyme compound with the antibody-antigen complex forms an antibody-antigen-antibody sandwich. After rinsing away unbound antibody-enzyme, a solution containing uncolored reagent is brought into contact with the antibody-antigen-antibody sandwich. The degree of color change indicates the amount of antigen in the test sample.

In competitive ELISA, there is provided a substrate having antigen bound to it, such as by covalent bonding. For example, the antigen may be bound to the inner surface of a well of a microtiter plate. A test sample is then combined with a first reagent containing antibody-enzyme conjugate to form a test mixture, an aliquot of which is then brought into contact with the substrate-bound antigen. Antigen bound to the substrate then displaces some antigen bound to antibody in the test mixture; the greater amount of antigen in the text mixture, the less antibody-enzyme conjugate will bind to the substrate-bound antigen to form an antigen-antibody-enzyme complex. After rinsing away unbound antibody-enzyme conjugate, the amount of antibody-enzyme conjugate bound to the substrate-bound antigen can be measured by contacting it with uncolored reagent and measuring the color change.

Other well-known immunological methods such as RadioImmunoAssay (RIA), western blotting, and immunofluorescence may also be used to detect the presence of an antigen of the invention in a test sample.

The present invention also provides a kit for carrying out an assay method according to the present invention. A kit according to the invention includes an antibody capable of binding to an antigen according to the present invention and at least one other component needed for carrying out the assay method. In some embodiments of the invention, the kit comprises a monoclonal anti-HCV short core peptide antibody.

Other components included in the kit according to the invention include adsorption media, buffered solutions and test kit instructions. In some embodiments, the antibody is linked to a detectable label, such as a radioactive label, a fluorescent label, a phosphorescent label, an enzyme, etc.

A kit of the invention for carrying out ELISA will contain an antibody of the invention covalently linked to a suitable enzyme capable of converting an uncolored reagent into its colored counterpart, and preferably in a separate sealed container, the uncolored reagent. Additionally, the kit may contain a substrate to which the antigen or antibody may be covalently or adsorbtively bound. Also, the kit may contain one or more solutions for rinsing away unbound antibody, instructions for using the kit, calibration tables or color charts, etc. In some embodiments, a kit of the invention includes an antibody pair as described above, wherein each of the antibodies binds with the same mitochondrially expressed HCV peptide. In particular embodiments, one such antibody is bound to a support, while another antibody is bound to a detectable label.

Thus, a kit for carrying out sandwich ELISA will include, at a minimum: a substrate to which is bound an antibody of the invention; in a separate container an antibody-enzyme conjugate; and in a further separate container an uncolored reagent which is converted into a colored species by the enzyme. As described above, in some embodiments of the invention the substrate-bound antibody and the enzyme-labeled antibody both bind the same mitochondrially expressed HCV peptide (albeit at different epitopes). Other kit components may include instructions for carrying out the assay method, rinsing solutions and calibration tables or charts, standard solutions for establishing a calibration curve, etc.

Thus, a kit for performing competitive ELISA according to the present invention will include, at a minimum: a substrate to which a peptide of the invention (SEQ ID NO:3, wherein Xaa is Arg or another amino acid,) is bound; in a separate container, a conjugate of an antibody to the peptide of the invention and an enzyme capable of converting an uncolored test reagent to a colored form; and, in a separate container, a solution containing the uncolored reagent. Other components of such a kit may include instructions for carrying out the method, a calibrated color chart for determining antigen concentration levels, rinsing solutions, standard solutions for establishing a calibration curve, etc.

Kits according to the invention for carrying out such methods will include, in separate containers, substrate and suitably labeled antibody. Other components that may be included in kits according to the invention include rinsing solutions, reagents, instructions for carrying out the methods, tables or charts of standard calibration curves, standard samples for establishing calibration curves, etc.

In some embodiments, a kit according to the invention includes at least two one monoclonal antibodies that binds to the same mitochondrially expressed HCV peptide having amino acids 1-39, wherein Xaa is Arg or another amino acid. In particular embodiments, the antibody is conjugated to an enzyme capable of converting a non-colored reagent to a colored reagent. In some embodiments, the kit according to the invention contains two or more antibodies according to the invention.

### EXAMPLES

### Example 1

### Production and Isolation of HCV Short Core Peptide

An epitope of HCV short core peptide (amino acids 21-40 of SEQ ID NO:1) was produced by immortalized cell line containing HCV within mitochondria.

A cell line from lymphocytes of a chronic hepatitis C patient has been established by immortalizing with Epstein-Barr virus. This cell line was named as SSP1 and described in detail in U.S. Patent Application (No. 60/583,945) and WO 2006/004768. This cell line has been deposited in ATCC (SD No. SD-5378). Cells were grown and mitochondria were fractionated as previously described in U.S. patent application.

Fractionated mitochondrial sample 5μg were electrophoresed by SDS 15% PAGE and cut in the middle of 20 kDa and 14 kDa markers. Higher molecular weight parts of the gels were used as positive control and lower parts were used for identification of bands. Then the gels were transferred to PVDF membranes. Blots were blocked by room temperature with 3% bovine serum albumin in PBST (phosphate buffered saline with 0.1% Tween-20) for 2 hours and then incubated overnight with mouse monoclonal anti-hepatitis C core antibody (Affinity Bioreagents, Golden, Colo.) at the dilution of 1:100 at 4° C. in PBST. After thorough washing, blots were incubated with anti-mouse antibodies conjugated to horseradish peroxidase (Amersham, Buckinghamshire, UK) in PBST. Detection was done by enhanced chemiluminescence (Pierce, Rockford, Ill.). Bands were identified at the position below the molecular weight marker of 14 kDa.

### Example 2

### Production and Isolation of HCV Short Core Peptide

HCV prime core epitope (SEQ ID NO:3; amino acids 1-39 of SEQ ID NO:1) produced by immortalized cell line described in Example 1.

This protein was theoretically designed and purchased from commercial manufacturers (Multiple Peptide Systems, San Diego, Calif.). By routine immunization protocol, rabbits were immunized to produce polyclonal antibody. Two hundred μg of peptide were injected in complete Freund's adjuvant. Rabbits were bled after three booster injections.

Samples prepared according to Example I were separated on 15% SDS-PAGE gels. Transferred to PVDF membrane and incubated with rabbit polyclonal antibody at 1:4,000 dilution at 4° C. overnight. After washing, blots were incubated with anti-rabbit antibodies conjugated with horseradish peroxidase (Amersham, Buckinghamshire, UK) at 1:20,000. The chemiluminescence detection system (Pierce, Rockford, Ill.) were employed according to manufacturer's instruction. At the position below the molecular weight marker of 4 kDa additional bands were observed.

### Example 3

### Generation of Monoclonal Antibodies Against HCV Short Core Peptide (SEQ ID NO:3)

Short core proteins were produced by conventional recombinant techniques. Several different cDNA were produced such as those encoding 1-39, 1-26, 14-39 of SEQ ID NO:1. Each cDNA was produced with primer extension method which were independent with viral DNA plate. The HCV complementary DNA fragment was inserted into the appropriate restriction sites of a commercially available GST (glutathione S-transferase) gene fusion vector, pGEX 2TK (Pharmacia). The vector construct was introduced to transform E. coli. Transformed bacteria were cultured in the 1: 10 2YTGA media for 1 hour. Subsequently, the expression of fusion protein was induced by addition of 0.10 mM IPTG (isopropyl-β-D-thiogalactopyranoside) for 1 hour. After sonication of the bacterial culture, the expressed protein was purified with glutathione-Sepharose beads at 1 μl of bed volume per 1 ml of culture ( Tong et al., J Virol. 1995; 69, 7106-7112). The purified protein is stored at −70° C. until it was used for immunization of mice to raise monoclonal antibodies.

Balb/c mice were immunized with short core peptides in complete Freund's adjuvant and injected subcutaneously or intraperitoneally in an amount from 1-100 μg. Alternatively, the peptides were emulsified in an adjuvant and injected in to the animal's hind foot pads. The immunized mice were then boosted 10 to 12 days later with additional peptides emulsified in the selected adjuvant. Thereafter, for several weeks, the mice were also be boosted with additional immunization injections. Serum samples were periodically obtained from the mice by retro-orbital bleeding for testing in ELISA assays to detect raised antibodies. After a suitable antibody titer is detected, the animals with the antibody reactivity can be injected with a final intravenous injection of the peptide. Three to four days later, the mice were sacrificed and the spleen cells were harvested. The spleen cells were then fused (using 35% polyethylene glycol) to a selected murine myeloma cell line. The fusion generated hybridoma cells, which were then plated in 96 well tissue culture plates containing HAT (hypoxanthine, aminopterin and thymidine) medium to inhibit proliferation of non-fused cells, myeloma hybrids and spleen cell hybrids. The hybridoma cells were then screened in an ELISA for reactivity against the used peptides. The hybridoma cells with positive reactivity were then injected intraperitoneally into syngenic Balb/c mice to produce ascites containing the monoclonal antibody. Purification of the monoclonal antibodies in the ascites were performed using ammonium sulfate precipitation, followed by gel exclusion chromatography. Alternatively, affinity chromatography based upon binding of antibody to protein A or Protein G were also used.

### Example 4

### A Kit for Detecting HCV in a Sample

A kit for detection of HCV includes, in separate containers:

An antibody directed toward an epitope of SEQ ID NO:4 bound to wells of a 96 well plate; an antibody bound to an enzyme; a reagent; and a wash buffer.

### Example 5

### Comparison of Reaction of Antibodies in Blood by ELISA

The table below shows the results of ELISA assays using various HCV-peptides as antigens. The first column shows the amino acids numbers of SEQ ID NO:1 that form the antigen. The second column presents the percent positive results for HCV-positive blood using the various peptides.

| Position of | % Positive in ELISA |
|---|---|
| SEQ ID NO: 1 | Sandwich Assay |
| 1-24 | 88% |
| 21-44 | 98 |
| 42-68 | 78 |
| 64-91 | 63 |
| 87-106 | 0 |
| 100-120 | 20 |
| 116-140 | 0 |
| 141-167 | 0 |
| 166-193 | 0 |

### Example 6

### Diagnosis of HCV Infection Using MAb to Mitochondrial Core Antigen

A monoclonal antibody (MAb) to SEQ ID NO:4 is prepared as described above. Using this method, HCV short core peptide is detected at a sensitivity of about 1 fg/ml.

Among hemodialysis patients, seroconversion of HCV antibody was prospectively examined to diagnose transfusion-related hepatitis C. Ten patients were diagnosed by the method of antibody detection (Abbott HCV EIA 2.0, Chicago, Ill.). Serial samples were tested with short core peptide ELISA described above. The results were compared with commercial RT-PCR method (Amplicor HCV 2.0, Roche, Pleasonton, Calif.). In 2 patients short core peptides were detected 3-4 weeks earlier than HCV RNA by RT-PCR.

All journal article, reference and patent citations provided above in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the following claims.

What is claimed is:

1. A method of detecting hepatitis C virus, comprising detecting a mitochondrially expressed HCV peptide.

2. The method of claim 1, wherein the mitochondrially expressed peptide is represented by SEQ ID NO:3, wherein Xaa is Arg or another amino acid.

3. The method of claim 1, wherein the mitochondrially expressed peptide is represented by SEQ ID NO:4.

4. An isolated peptide consisting of the amino acid sequence represented by SEQ ID NO:3, wherein Xaa is Arg or another amino acid residue.

5. The isolated peptide of claim 4 having the sequence of SEQ ID NO:4.

6. The peptide of claim 4, wherein said peptide is mitochondrially expressed.

7. A method of making an antibody to the peptide of SEQ ID NO:3, comprising:
(a) introducing a HCV short core peptide of SEQ ID NO:3 into an animal;
(b) isolating splenocytes from the animal;
(c) fusing the splenocytes with immortalized cells to form hybridomas; and
(d) isolating said antibody to the peptide of SEQ ID NO:3.

8. An antibody that specifically binds to the peptide of claim 4.

9. A method of detecting HCV, comprising detecting the presence of a peptide consisting of the amino acid sequence of SEQ ID NO:3,wherein Xaa is Arg or another amino acid residue.

10. A method of detecting HCV, comprising detecting the presence of a mitochondrially expressed peptide consisting of the amino acid sequence of SEQ ID NO:3, wherein Xaa is Arg or another amino acid residue.

11. A method of detecting HCV in a sample, comprising:
(a) providing a support-bound antibody that binds a first epitope of a mitochondrially expressed peptide of SEQ ID NO:3, wherein the first antibody is immobilized on a support medium;
(b) contacting the support-bound antibody with a sample, whereby mitochondrially expressed peptide of SEQ ID NO:3 binds to the support-bound antibody to form an antibody-antigen complex;
(c) contacting the support-bound antibody with a labeled antibody that binds a second epitope of the mitochondrially expressed peptide of SEQ ID NO:3, whereby labeled antibody binds to the mitochondrially expressed peptide of SEQ ID NO:3 to form an antibody-antigen-antibody complex; and
(d) detecting the antibody-antigen-antibody complex, whereby detection of antibody-antigen-antibody complex above a predetermined threshold indicates presence of HCV in the sample.

12. A method of detecting the presence of HCV in a subject, comprising:
(a) providing a support-bound antibody that binds a first epitope of a mitochondrially expressed peptide of SEQ ID NO:3, wherein the first antibody is immobilized on a support medium;
(b) contacting the support-bound antibody with a sample from the subject, whereby mitochondrially expressed peptide of SEQ ID NO:3 binds to the support-bound antibody to form an antibody-antigen complex;
(c) contacting the support-bound antibody with a labeled antibody that binds a second epitope of the mitochondrially expressed peptide of SEQ ID NO:3, whereby labeled antibody binds to the mitochondrially expressed peptide of SEQ ID NO:3 to form an antibody-antigen-antibody complex; and
(d) detecting the antibody-antigen-antibody complex, whereby detection of antibody-antigen-antibody complex above a predetermined threshold indicates presence of HCV in the subject.

13. The method of claim 11 or 12, wherein the sample is blood.

14. The method of claim 11 or 12, wherein the sample is human blood.

15. The method of claim 11 or 12, wherein the sample is from a subject that is asymptomatic of HCV infection.

16. A kit comprising:
(a) a support-bound antibody that binds a first epitope of a mitochondrially expressed HCV peptide of SEQ ID NO:3; and
(b) a labeled antibody that binds a second epitope of a mitochondrially expressed HCV peptide of SEQ ID NO:3.

17. The kit of claim 16, further comprising a rinsing reagent.


SEQUENCE LISTING (4 sequences)

SEQ ID NO: 1 - protein, 40 aa, Hepatitis C Virus
MSTNPKPQRKTKRNTNRRPQDVKFPGGGQIVGGVYLLPRR

SEQ ID NO: 2 - DNA, 120 bp, Hepatitis C Virus
catgagcacgaatcctaaacctcaaagaaaaaccaaacgtaacaccaaccgtcgcccacaggacgtcaagttcccgggtggcggtcagatcgttggtggagtttacttgttgccgcgcag

SEQ ID NO: 3 - protein, 39 aa, Hepatitis C Virus
  VARIANT 9: Xaa = Arg or another amino acid
MSTNPKPQXKTKRNTNRRPQDVKFPGGGQIVGGVYLLPR

SEQ ID NO: 4 - protein, 39 aa, Hepatitis C Virus
MSTNPKPNRKTKRNTNRRPQDVKFPGGGQIVGGVYLLPR